(19) United States
(12) Patent Application Publication
(10) Pub. No.: US 2011/0230769 A1
(43) Pub. Date: Sep. 22, 2011

(54) Information processing apparatus and information processing method

(75) Inventor: Takayuki Yamazaki, Aichi (JP)

(73) Assignee: Sony Corporation, Tokyo (JP)

(21) Appl. No.: 12/932,437

(22) Filed: Feb. 25, 2011

(30) Foreign Application Priority Data
Mar. 17, 2010 (JP) P2010-061168

(51) Int. Cl. A61B 6/00

(52) U.S. Cl. 600/473

(57) ABSTRACT

An information processing apparatus for performing authentication using veins of a living body part, the information processing apparatus includes: a visible light source configured to present through light emission the position on which to place the living body part; a light-receiving section configured to receive reflected light of the visible light from the visible light source; a computation section configured to compute the amount of misalignment of the living body part with the placement position on the basis of the intensity of the reflected light received by the light-receiving section; and a control section configured to prompt correction of the placement of the living body part for alignment with the placement position in accordance with the misalignment amount computed by the computation section.

### CROSS-REFERENCE TO RELATED APPLICATION

The present application claims priority from Japanese Patent Application No. JP 2010-061168 filed in the Japanese Patent Office on Mar. 17, 2010, the entire content of which is incorporated herein by reference.

### BACKGROUND OF THE INVENTION

1. Field of the Invention

The present invention relates to an information processing apparatus and an information processing method. More particularly, the invention relates to an information processing apparatus and an information processing method for performing authentication accurately in a biometric authentication process even where the position for living body part placement is flat.

2. Description of the Related Art

In recent years, there have been known biometric authentication apparatuses for authenticating individuals using the veins of their fingers or their palms.

In order to perform authentication accurately with such a biometric authentication apparatus, it is necessary for the user precisely to place his or her palm or finger onto the position in which to read (i.e., image) the venous pattern of the body part (the position may be called the placement position hereunder).

Some apparatuses are arranged to illuminate the surroundings of the position for finger placement so that the user may know the exact position to put his or her finger on (see Japanese Patent Laid-Open No. 2005-323892).

The arrangement above allows the user to determine clearly where the placement position is and thus to put his or her finger precisely onto the placement position.

However, this technique does not envisage sensing the position where the user's finger is actually placed. The user thus fails to notice any possible misalignment of the fingertips with the accurate placement position. This can make it difficult to perform authentication with precision.

There exists a technique which, if the user has failed to put his or her finger preciously onto the placement position, allows the image taken of the venous pattern to be corrected to permit accurate authentication. More specifically, the technique involves emitting light beams of different wavelengths at the user's finger to acquire a fingerprint image and a venous pattern image. If the user's finger is not aligned with the placement position, a misalignment is detected from the fingerprint image and the detected misalignment is used as the basis for correcting the venous pattern image (see Japanese Patent Laid-Open No. 2006-72764, called the Patent Document 1 hereunder).

### SUMMARY OF THE INVENTION

According to the technique of the Patent Document 1, the placement position for the finger is formed as guide grooves so that the user's finger will not be misaligned substantially from the correct placement position. However, if the placement position for the finger is formed as a flat shape based on this technique, the user finds it difficult to determine the correct placement position. This poses the possibility that the user's finger will be so misaligned with the placement position as to make it impossible to correct the venous pattern image in accordance with the misalignment. As a result, authentication may not be carried out precisely.

The present invention has been made in view of the above circumstances and provides arrangements for performing authentication accurately in a biometric authentication process even where the position for living body part placement is flat.

In carrying out the present invention and according to one embodiment thereof, there is provided an information processing apparatus for performing authentication using veins of a living body part, the information processing apparatus including: a visible light source configured to present through light emission the position on which to place the living body part; a light-receiving section configured to receive reflected light of the visible light from the visible light source; a computation section configured to compute the amount of misalignment of the living body part with the placement position on the basis of the intensity of the reflected light received by the light-receiving section; and a control section configured to prompt the correction of the placement of the living body part for alignment with the placement position in accordance with the misalignment amount computed by the computation section.

Preferably, the control section may prompt the correction of the placement of the living body part for alignment with the placement position by controlling the emission of the visible light in accordance with the misalignment amount computed by the computation section.

Preferably, the information processing apparatus may further include: a near-infrared light source configured to emit near-infrared light to the living body part; and an imaging section configured to take an image of the living body part to which the near-infrared light is emitted; wherein the computation section may compute the misalignment amount based on the intensity of the reflected light received by the light-receiving section and on the image of the living body part taken by the imaging section.

Preferably, if the misalignment amount is larger than a predetermined threshold value, then the control section may prompt the correction of the placement of the living body part for alignment with the placement position.

Preferably, the information processing apparatus may further include an imaging control portion configured to adjust imaging parameters of the imaging section if the misalignment amount is smaller than the predetermined threshold value.

Preferably, the information processing apparatus may further include a determination section configured to determine whether an object imaged by the imaging section is the living body part; wherein, if the determination section determines that the object is the living body part, then the computation section may compute the misalignment amount.

Preferably, the information processing apparatus may further include a recording section configured to record the image taken by the imaging section upon user registration; wherein the computation section may compute the misalignment amount based on the intensity of the reflected light received by the light-receiving section and on a difference between the image taken by the imaging section and the image recorded in the recording section.

Preferably, the information processing apparatus may further include a display section configured to display a predetermined image or text; wherein the control section may cause the display section to display an image or a text prompting the correction of the placement of the living body part for alignment with the placement position in accordance with the misalignment amount computed by the computation section.

Preferably, the information processing apparatus may further include a sound output section configured to output a sound; wherein the control section may cause the sound output section to output a sound prompting the correction of the placement of the living body part for alignment with the placement position in accordance with the misalignment amount computed by the computation section.

Preferably, the information processing apparatus may further include a temperature difference generation section configured to generate a temperature difference near the placement position; wherein the control section may cause the temperature difference generation section to generate a temperature difference prompting the correction of the placement of the living body part for alignment with the placement position in accordance with the misalignment amount computed by the computation section.

Preferably, the information processing apparatus may further include a vibration generation section configured to generate vibrations near the placement position; wherein the control section may cause the vibration generation section to generate vibrations prompting the correction of the placement of the living body part for alignment with the placement position in accordance with the misalignment amount computed by the computation section.

Preferably, the information processing apparatus may further include: a display section configured to display a predetermined image or text; a sound output section configured to output a sound; a temperature difference generation section configured to generate a temperature difference near the placement position; and a vibration generation section configured to generate vibrations near the placement position. The control section may cause the visible light source to emit the light prompting the correction of the placement of the living body part for alignment with the placement position in accordance with the misalignment amount computed by the computation section. The control section may cause the display section to display an image or a text prompting the correction of the placement of the living body part for alignment with the placement position in accordance with the misalignment amount. The control section may cause the sound output section to output a sound prompting the correction of the placement of the living body part for alignment with the placement position in accordance with the misalignment amount. The control section may cause the temperature difference generation section to generate a temperature difference prompting the correction of the placement of the living body part for alignment with the placement position in accordance with the misalignment amount. The control section may cause the vibration generation section to generate vibrations prompting the correction of the placement of the living body part for alignment with the placement position in accordance with the misalignment amount.

Preferably, the living body part mentioned above may be a human finger.

According to another embodiment of the present invention, there is provided an information processing method for use with an information processing apparatus which performs authentication using veins of a living body part and which includes a visible light source configured to present through light emission the position on which to place the living body part and a light-receiving section configured to receive reflected light of the visible light from the visible light source, the information processing method including the steps of: computing the amount of misalignment of the living body part with the placement position on the basis of the intensity of the reflected light received by the light-receiving section; and performing control to prompt correction of the placement of the living body part for alignment with the placement position in accordance with the misalignment amount computed by the computation section.

According to the present invention embodied as outlined above, the amount of misalignment of the living body part with the placement position is computed on the basis of the intensity of the reflected light received by the light-receiving section. Then a prompt is made to correct the placement of the living body part for alignment with the placement position in accordance with the computed misalignment amount.

Thus according to the present invention outlined above, it is possible to perform authentication accurately in a biometric authentication process even where the position for living body part placement is flat.

### BRIEF DESCRIPTION OF THE DRAWINGS

FIG. 1 is a schematic view showing a typical external structure of an authentication unit as an embodiment of the present invention;

FIG. 2 is a block diagram showing a typical functional structure of the authentication unit;

FIG. 3 is a schematic view explanatory of a block layout inside the authentication unit;

FIG. 4 is a flowchart explanatory of a registration process performed by the authentication unit;

FIG. 5 is a schematic view showing a typical display of the position for finger placement;

FIG. 6 is a flowchart explanatory of an authentication process performed by the authentication unit;

FIG. 7 is a flowchart explanatory of a misalignment notification process performed by the authentication unit;

FIG. 8 is a schematic view explanatory of misalignment of a finger with the finger placement position;

FIG. 9 is a schematic view explanatory of an example in which a prompt is made to correct the placement of the finger for alignment with the finger placement position;

FIG. 10 is a schematic view explanatory of another example in which a prompt is made to correct the placement of the finger for alignment with the finger placement position;

FIG. 11 is a schematic view explanatory of a further example in which a prompt is made to correct the placement of the finger for alignment with the finger placement position;

FIG. 12 is a schematic view explanatory of an even further example in which a prompt is made to correct the placement of the finger for alignment with the finger placement position;

FIG. 13 is a schematic view explanatory of a still further example in which a prompt is made to correct the placement of the finger for alignment with the finger placement position;

FIG. 14 is a block diagram showing another typical functional structure of the authentication unit;

FIG. 15 is a schematic view showing a typical external structure of a notebook-size personal computer equipped with the authentication unit according to the present invention;

FIG. 16 is a block diagram showing a further typical functional structure of the authentication unit;

FIG. 17 is a block diagram showing an even further typical functional structure of the authentication unit; and

FIG. 18 is a block diagram showing a still further typical functional structure of the authentication unit.

### DETAILED DESCRIPTION OF THE PREFERRED EMBODIMENTS

Some preferred embodiments of the present invention will now be explained in reference to the accompanying drawings.

### [External Structure of the Authentication Unit]

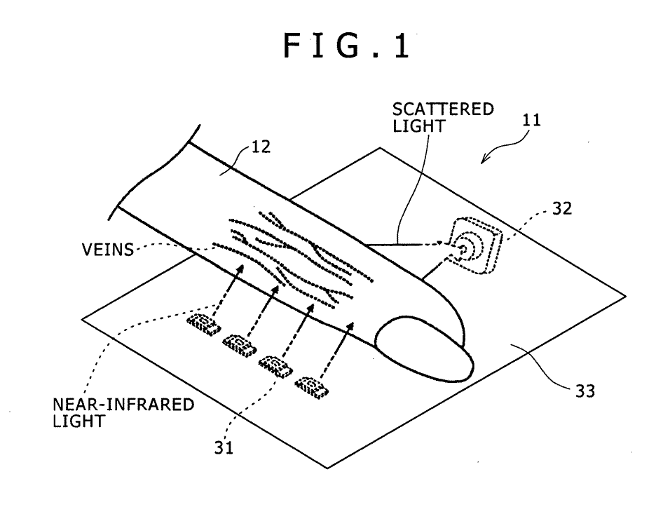

FIG. 1 shows a typical external structure of an authentication unit 11 as an embodiment of the present invention.

The authentication unit 11 shown in FIG. 1 performs venous pattern authentication of a finger 12 that is a living body part.

More specifically, in the authentication unit 11, a near-infrared light source 31 emits near-infrared light to the finger 12 while an imaging section 32 images the light scattered by the finger 12. That is, the imaging section 32 takes an image of a venous pattern formed by the hemoglobin inside those veins of the finger 12 which absorb the near-infrared light from the near-infrared light source 31.

The near-infrared light source 31 is composed of LED's (light emitting diodes) emitting light in an infrared spectrum part ranging from about 0.7 to 2.5 μm. The near-infrared light source 31 is formed by a plurality of LED's arrayed linearly in the lengthwise direction of the finger 12. The LED's constituting the near-infrared light source 31 are arrayed at suitable lighting angles so that excess near-infrared light will not spill into the imaging section 32 and that only the finger 12 will be lit in the position for taking an image of the venous pattern of the finger 12.

The imaging section 32 is made up of an optical block and a photoelectric conversion element such as a CCD (charge coupled device) or CMOS (complementary metal-oxide semiconductor). The optical block forms an optical image of an object. The photoelectric conversion element acquires the image of the object by converting the optical image (i.e., image of the object) into image data that is an electrical signal.

In the authentication unit 11 of FIG. 1, the near-infrared light source 31 and imaging section 32 are disposed under a transmission filter 33. The transmission filter 33 is formed in such a manner that both sides thereof have a flat shape. The material from which the transmission filter 33 is made lets the near-infrared light from the near-infrared light source 31 and the scattered light from the finger 12 pass through. In addition to the near-infrared light from the near-infrared light source 31 and the scattered light from the finger 12, the transmission filter 33 lets the visible light from a visible light source, to be discussed later, and reflected light of the visible light from the finger 12 pass through.

In the authentication unit 11 of FIG. 1, the near-infrared light source 31 emits near-infrared light diagonally and unidirectionally to the finger 12 placed on the finger placement position on the topside of the transmission filter 33. The imaging section 32 then images the venous pattern of the finger 12. Thus the authentication unit 11 may be constructed in such a manner that the near-infrared light source 31 and imaging section 32 are disposed on the same plane under the transmission filter 33.

In that structure, the authentication unit 11 on the side of the finger 12 (i.e., topside of the transmission filter 33) may be shaped flat. Also, the authentication unit 11 as a whole can be formed into a thin shape.

In the above-described structure, the authentication unit 11 performs venous pattern authentication by collating an image taken of the venous pattern with the venous pattern imaged and recorded upon registration.

### [Typical Functional Structure of the Authentication Unit]

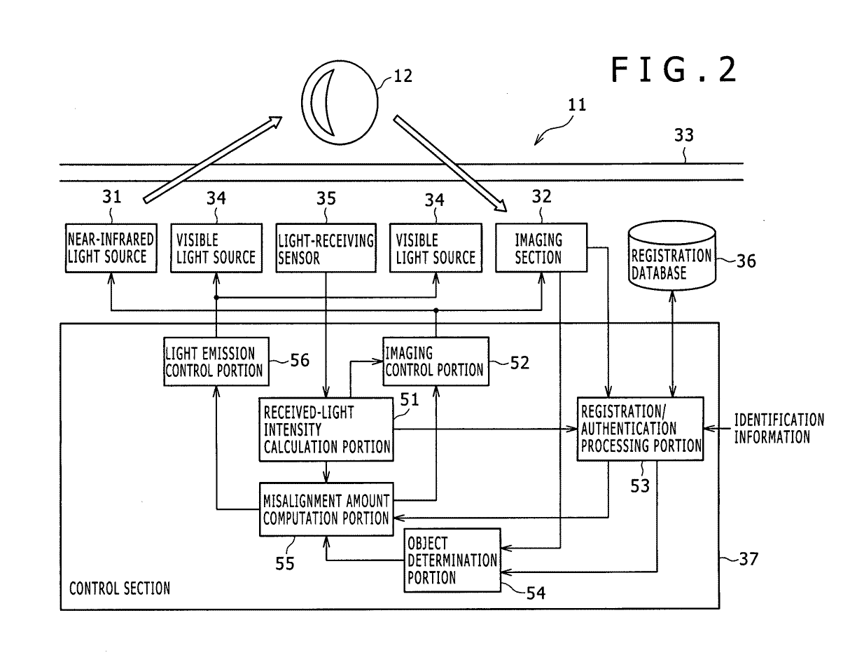

A typical functional structure of the authentication unit 11 will now be explained in reference to FIG. 2.

The authentication unit 11 shown in FIG. 2 is made up of the near-infrared light source 31, the imaging section 32, the transmission filter 33, a visible light source 34, a light-receiving sensor 35, a registration database (DB) 36, and a control section 37.

Also, the finger 12 in FIG. 2 is shown at the angle at which the finger 12 in FIG. 1 is viewed from the fingertip. The near-infrared light source 31, visible light source 34, and light-receiving sensor 35 are shown in a cross-sectional view taken at that angle in the authentication unit 11. That is, in the authentication unit 11, the near-infrared light source 31, imaging section 32, visible light source 34, and light-receiving sensor 35 are disposed (mounted) on the same plane (on the same substrate) under the transmission filter 33.

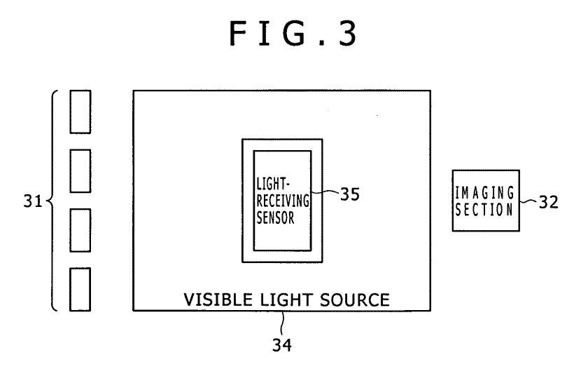

A typical layout of the near-infrared light source 31, imaging section 32, visible light source 34, and light-receiving sensor 35 are now explained below in reference to FIG. 3.

FIG. 3 is a top view of the authentication unit 11 excluding the transmission filter 33. The bottom side of FIG. 3 corresponds to the tip side of the finger 12 shown in FIG. 1.

As shown in FIG. 3, the visible light source 34 having a predetermined area is interposed between the near-infrared light source 31 and the imaging section 32. The visible light source 34 is shaped to have a hollow center occupied by the light-receiving sensor 35. The light-receiving sensor 35 is laid out in such a manner that the position opposed to the light-receiving sensor 35 across the transmission filter 33 (i.e., immediately above the light-receiving sensor 35) is the position on which to place the finger 12.

The layout of the near-infrared light source 31 and visible light source 34 is not limited to the one shown in FIG. 3. Any other layout will do as long as the imaging section 32 can take an image of the scattered light from the finger 12 and the light-receiving sensor 35 can receive reflected light from the finger 12 in the layout in question.

Referring back to the explanation in reference to FIG. 2, the near-infrared light source 31, imaging section 32, and transmission filter 33 shown in FIG. 2 are the same as the near-infrared light source 31, imaging section 32, and transmission filter 33 described above in reference to FIG. 1. Thus their descriptions will not be repeated hereunder.

The visible light source 34 is composed of a plurality of LED's emitting visible light in a visible light spectrum part ranging from about 380 to 750 nm. The visible light source 34 is structured with a plurality of LED's arrayed in the region shown in FIG. 3. The visible light emitted by the visible light source 34 passes through the transmission filter 33 to present the user with the placement position for the finger 12 while lighting the finger 12.

The light-receiving sensor 35 receives reflected light of the visible light that was emitted to and reflected from the finger 12 before passing through the transmission filter 33. The light-receiving sensor 35 supplies the control section 37 with information representing the level of the received light.

The registration database 36 is typically composed of a hard disk or a nonvolatile memory. As such, the registration database 36 records user information supplied from the control section 37 for user authentication. The user information recorded in the registration database 36 is rewritable and is retrieved as needed by the control section 37.

The control section 37 is made up of a CPU (central processing unit), a ROM (read only memory), and a RAM (random access memory). The control section 37 controls the components of the authentication unit 11.

The control section 37 includes a received-light intensity calculation portion 51, an imaging control portion 52, a registration/authentication processing portion 53, an object determination portion 54, a misalignment amount computation portion 55, and a light emission control portion 56.

The received-light intensity calculation portion 51 calculates the intensity of the reflected light received from the finger 12 on the basis of received-light level information coming from the light-receiving sensor 35. The received-light intensity thus calculated is fed to the registration/authentication processing portion 53 or misalignment amount computation portion 55. Also, upon detection of a change in received-light intensity typically as a result of the finger 12 getting placed on the transmission filter 33, the received-light intensity calculation portion 51 supplies the imaging control portion 52 with information giving an instruction to start imaging.

The imaging control portion 52 controls the near-infrared light source 31 and imaging section 32 in operation. For example, when supplied from the received-light intensity calculation portion 51 with information giving the instruction to start imaging, the imaging control portion 52 causes the near-infrared light source 31 to emit near-infrared light and the imaging section 32 to start imaging. The image data (simply called the image hereunder) acquired by the imaging section 32 is sent to the registration/authentication processing portion 53 or object determination portion 54 under control of the imaging control portion 52.

The registration/authentication processing portion performs registration and authentication of user information in the authentication unit 11.

More specifically, when the authentication unit 11 is in registration mode, the registration/authentication processing portion 53 establishes correspondences among the identification information for user identification coming from an input section, not shown, the image of the finger 12 from the imaging section 32, and the received-light intensity from the received-light intensity calculation portion 51, on the basis of the identification information. The items of information thus made in correspondence with one another are supplied and written to the registration database 36 as user information.

Also, when the authentication unit 11 is in authentication mode, the registration/authentication processing portion 53 reads corresponding user information from the registration database 36 based on the identification information supplied from the input section, not shown. The registration/authentication processing portion 53 proceeds to collate the finger image and the received-light intensity of the user information with the finger image coming from the imaging section 32 and with the received-light intensity from the received-light intensity calculation portion 51, respectively.

When the authentication unit 11 is in authentication mode, the object determination portion 54 determines whether the object of the image coming from the imaging section 32 is a human finger. If the object of the image from the imaging section 32 turns out to be a human finger, the object determination portion 54 acquires the user information from the registration/authentication processing portion 53, and compares in size the finger image of the user information with the finger image from the imaging section 32. The object determination portion 54 proceeds to supply the misalignment amount computation portion 55 with information representing the difference in size obtained from the comparison between the two finger images.

Based on the received-light intensity fed from the received-light intensity calculation portion 51 and on the difference in size between finger images from the object determination portion 54, the misalignment amount computation portion 55 computes the amount of misalignment (called the misalignment amount hereunder) of the finger 12 with the placement position on the transmission filter 33, and sends the computed misalignment amount to the light emission control portion 56.

The light emission control portion 56 controls the light emission of the visible light source 34 in accordance with the misalignment amount coming from the misalignment amount computation portion 55. Depending on the misalignment amount from the misalignment amount computation portion 55, the light emission control portion 56 prompts the visible light source 34 to emit visible light in order to let the placement of the finger 12 be corrected for alignment with the placement position.

### [User Registration Process Performed by the Authentication Unit]

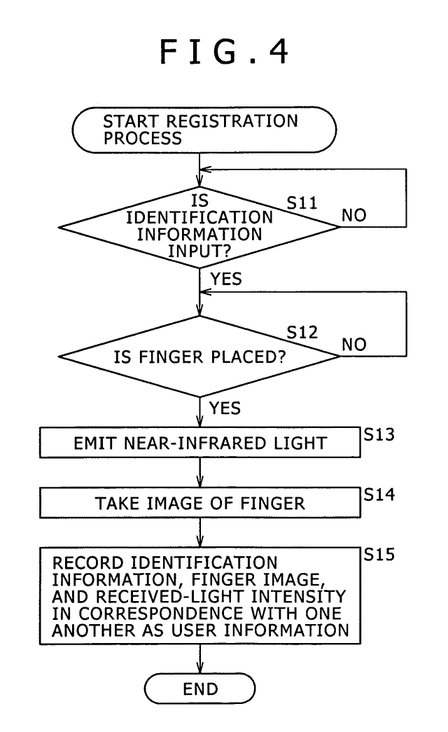

The user registration process performed by the authentication unit 11 will now be explained in reference to the flowchart of FIG. 4.

The registration process shown in the flowchart of FIG. 4 is carried out by the authentication unit 11 when the authentication unit 11 goes into registration mode from an operation mode as a result of the user operating the input section, not shown.

In step S11, the registration/authentication processing portion 53 determines whether the identification information for identifying the user is input through the input section, not shown. If the input section is structured as, say, a numerical keypad, the identification information may be an ID number entered by the user through the numerical keypad. If the input section is structured as a card reader, then the identification information may be a user ID recorded on an ID card owned by the user.

If it is determined in step S11 that identification information is not input yet, step S11 is repeated until identification information is input. If it is determined in step S11 that identification information is input, then control is transferred to step S12.

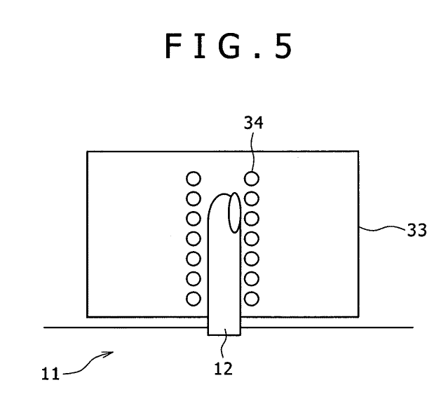

At this point, as shown in FIG. 5, the light emission control portion 56 controls the visible light source in visible light emission so that the placement position for the finger 12 on the transmission filter 33 is presented to the user. The geometry of the emission by the visible light source 34 indicating the placement position for the finger 12 is not limited to what is shown in FIG. 5. Any other geometry of the emission will do as long as it allows the user to recognize the placement position for the finger 12, including one that highlights the contour of the finger 12.

In step S12, the received-light intensity calculation portion 51 determines whether the finger 12 is placed on the placement position on the transmission filter 33 based on the information from the light-receiving sensor 35 indicating the received-light level.

If it is determined in step S12 that the finger 12 is not placed on the placement position on the transmission filter 33, i.e., if it is determined that there is no change in the received-light level from the light-receiving sensor 35 according to the information therefrom indicating the received-light level, then step S12 is repeated until there occurs a change in the received-light level from the light-receiving sensor 35.

If it is determined in step S12 that the finger 12 is placed on the placement position on the transmission filter 33, i.e., if it is determined that the received-light level from the light-receiving sensor 35 is raised abruptly by the finger getting placed onto the placement position on the transmission filter 33 according to the received-light level information from the light-receiving sensor 35, then the received-light intensity calculation portion 51 supplies the imaging control portion 52 with information giving an instruction to start imaging. Also, based on the received-light level information from the light-receiving sensor 35, the received-light intensity calculation portion 51 calculates the intensity of the reflected light from the finger 12 and feeds the calculated received-light intensity to the registration/authentication processing portion 53. Thereafter, control is transferred to step S13.

In step S13, the imaging control portion 52 causes the near-infrared light source 31 to emit near-infrared light based on the information from the received-light intensity calculation portion 51 giving the instruction to start imaging. The near-infrared light is emitted to the finger 12 placed on the placement position on the transmission filter 33.

In step S14, based on the information from the received-light intensity calculation portion 51 giving the instruction to start imaging, the imaging control portion 52 causes the imaging section 32 to take an image of the finger 12 which is placed on the placement position and to which the near-infrared light is emitted. More specifically, when supplied from the received-light intensity calculation portion with the information giving the instruction to start imaging, the imaging control portion 52 causes the imaging section 32 to start imaging the finger 12. The imaging control portion 52 causes the imaging section 32 to supply the registration/authentication processing portion 53 with the finger image acquired upon elapse of a predetermined time period.

In step S15, the registration/authentication processing portion 53 establishes correspondences among the identification information input through the input section, not shown, the finger image from the imaging section 32, and the received-light intensity from the received-light intensity calculation portion 51. These items of information made in correspondence with one another are supplied and written to the registration database 36 as user information.

When the above steps have been carried out, the user placing his or her finger 12 onto the placement position of the transmission filter 33 can have his or her user information registered.

### [User Authentication Process Performed by the Authentication Unit]

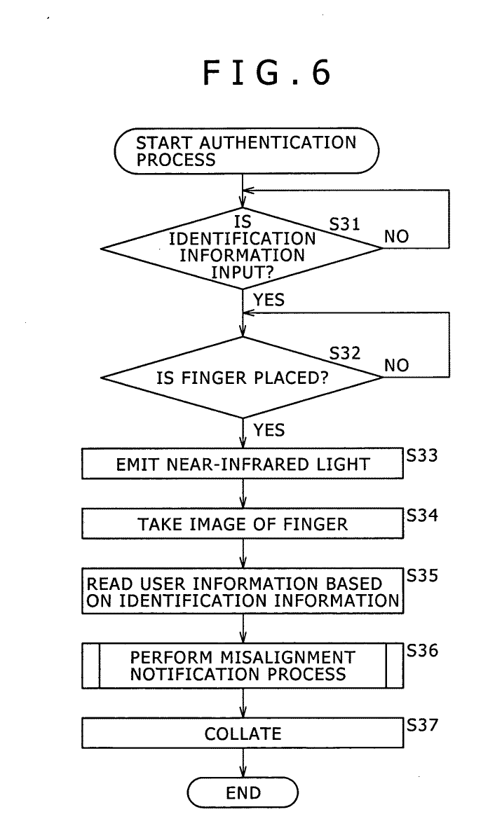

Explained next in reference to the flowchart of FIG. 6 is the user authentication process performed by the authentication unit 11.

The authentication process shown in the flowchart of FIG. 6 is carried out when the authentication unit 11 goes into authentication mode from an operation mode as a result of the user operating the input section, not shown.

Steps S31 through S34 in the flowchart of FIG. 6 are the same as steps S11 through S14 explained above in reference to the flowchart in FIG. 4 and thus will not be described further in detail. The received-light intensity calculated when the finger 12 is placed on the placement position in step S32 is sent to the misalignment amount computation portion 55, and the finger image taken in step S34 is fed to the object determination portion 54.

In step S35, based on the identification information input through the input section, not shown, the registration/authentication processing portion 53 searches the registration database 36 for the user information corresponding to the input identification information and retrieves the corresponding user information.

In step S36, the authentication unit 11 performs a misalignment notification process notifying the user of any misalignment of the finger 12 that may occur with the placement position on the transmission filter 33.

### [Misalignment Notification Process Performed by the Authentication Unit]

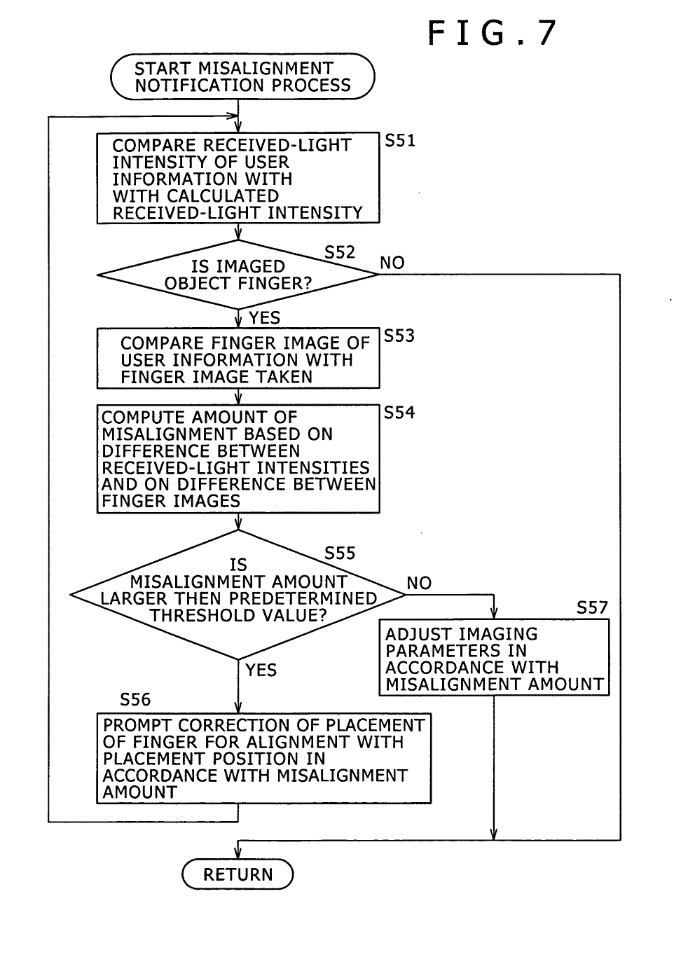

The misalignment notification process performed by the authentication unit 11 is explained below in reference to the flowchart in FIG. 7.

In step S51, the misalignment amount computation portion 55 acquires the received-light intensity of the user information retrieved by the registration/authentication processing portion 53, and compares the received-light intensity of the user information with the received-light intensity coming from the received-light intensity calculation portion 51 to find a difference therebetween.

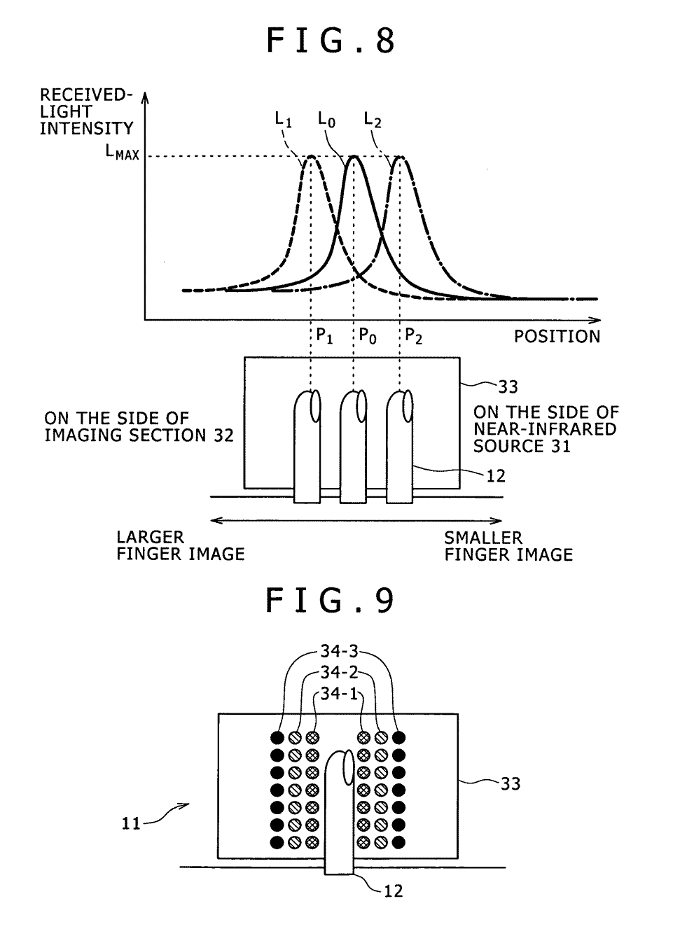

FIG. 8 is a schematic view explanatory of typical received-light intensities that are compared with one another. FIG. 8 shows relationship between the placement position of the finger 12 on the transmission filter 33 on the one hand and received-light intensities on the other hand.

In FIG. 8, a received-light intensity curve L0 indicated by a solid line represents the received-light intensity recorded in the registration database 36 as user information; and a position P0 denotes the placement position for the finger 12 in effect when the received-light intensity represented by the received-light intensity curve L0 (the intensity is called the received-light intensity L0 hereunder) is obtained. At the position P0, the received-light intensity L0 takes a peak value LMAX.

Received-light intensities L1 and L2 indicated by a broken line and a dashed line respectively are typical of the received-light levels fed from the received-light intensity calculation portion 51 in authentication mode. Positions P1 and P2 represent the placement positions for the finger 12 in effect when the received-light intensities indicated by the received-light intensity curves L1 and L2 respectively (called the received-light intensities L1 and L2 hereunder) are obtained. At the positions P1 and P2, the received-light intensities L1 and L2 each take the peak value LMAX.

For example, when the received-light intensity L1 is supplied from the received-light intensity calculation portion 51, the misalignment amount computation portion 55 compares the position P0 at which the received-light intensity L0 of the user information takes the peak value LMAX, with the position P1 at which the peak value LMAX of the received-light intensity L1 is obtained by the received-light intensity calculation portion 51, to find a difference therebetween.

Referring back to the flowchart of FIG. 7, in step S52, the object determination portion 54 determines whether the object of the image taken by the imaging section 32 is a human finger. The determination of whether the object is a human finger may be performed typically by comparing a prepared template image with the object in question or by carrying out other suitable image processing.

If it is determined in step S52 that the imaged object is a human finger, then control is transferred to step S53.

In step S53, the object determination portion 54 acquires the finger image of the user information retrieved by the registration/authentication processing portion 53, and compares the finger image of the user information with the finger image determined to be representative of a human finger to find a difference therebetween.

For example, if the finger 12 is placed on the position P1 on the transmission filter 33 shown in FIG. 8, the finger image at the position P1 appears larger than the finger image of the user information acquired when the finger 12 is placed on the position P0. That is because the position P1 is closer to the imaging section 32 than the position P0.

If the finger 12 is placed on the position P2 on the transmission filter 33 shown in FIG. 8, the finger image at the position P2 appears smaller than the finger image of the user information acquired when the finger 12 is placed on the position P0. That is because the position P2 is farther from the imaging section 32 than the position P0.

Thus the object determination portion 54 may typically compare the finger width in the finger image of the user information with the finger width in the finger image taken by the imaging section 32 to find a difference therebetween. The acquired difference is sent to the misalignment amount computation portion 55.

In step S54, the misalignment amount computation portion 55 computes the amount of misalignment of the finger 12 with the placement position on the transmission filter 33 based on the difference between the received-light intensities obtained in step S51 and on the difference between the finger images supplied from the object determination portion 54.

More specifically, the misalignment amount computation portion 55 computes the amount of misalignment of the finger 12 with the placement position on the transmission filter 33 (the amount may also be called the actual misalignment amount hereunder) on the basis of, say, the difference between the positions P0 and P1 shown in FIG. 8.

For example, if the light-receiving sensor 35 receives the reflected light from the finger 12 in units of pixels, then the received-light intensity calculation portion calculates the intensity of the received light also in units of pixels. Thus the difference between the positions P0 and P1 shown in FIG. 8 is obtained in units of pixels. For example, if a misalignment amount of 100 μm on the transmission filter 33 corresponds to one pixel of the light-receiving sensor 35, then a difference of 1,000 pixels between the positions P0 and P1 causes the imaging section 32 to acquire 10 mm of actual misalignment amount of the finger 12 with regard to the placement position on the transmission filter 33.

Also, the misalignment amount computation portion 55 computes the amount of relative misalignment (also called the relative misalignment amount hereunder) corresponding to the amount of misalignment of the finger 12 with the placement position on the transmission filter 33 based on the difference between the finger width in the finger image of the user information and the finger width in the finger image taken by the imaging section 32.

For example, if the finger width in the finger image taken by the imaging section 32 is 750 pixels and if the finger width in the finger image of the user information is 500 pixels, the difference of 250 pixels constitutes the relative misalignment amount. In this case, the finger width in the finger image of the user information is less than the finger with in the finger image taken by the imaging section 32. That means the finger 12 is shifted from the placement position towards the near-infrared light source 31.

In step S55, the misalignment amount computation portion 55 determines whether the misalignment amount computed in step S54 is larger than a predetermined threshold value. More specifically, the misalignment amount computation portion 55 determines whether the actual misalignment amount and the relative misalignment amount are each larger than a predetermined corresponding threshold value.

If it is assumed here that the predetermined threshold value for the actual misalignment amount is 8 mm and that the predetermined threshold value for the relative misalignment amount is 200 pixels, then it is determined in step S55 of the above example that the misalignment amount is larger than the threshold value. In this case, the misalignment amount computation portion 55 supplies the light emission control portion 56 with information representative of the calculated amount of misalignment (actual misalignment amount). Control is then transferred to step S56.

The threshold value for the amount of misalignment may be established variably depending on the security level demanded. For example, the threshold value may be set low for the authentication permitting entry into buildings, and set high for the authentication permitting access to ATM's (automated teller machines) at banks or like institutions.

In step S56, the light emission control portion 56 controls the light emission of the visible light source 34 in accordance with the information representative of the misalignment amount coming from the misalignment amount computation portion 55. Through such light emission control, the light emission control portion 56 prompts the correction of the placement of the finger 12 on the placement position.

As shown in FIG. 9 for example, the light emission control portion 56 causes a plurality of rows (two in FIG. 2) of visible light source elements 34-2 and 34-3 to emit light outside of visible light source elements 34-1 (i.e., visible light source 34) representing the placement position shown in FIG. 5. One of the multiple rows of visible light source elements 34-1 through 34-3 is caused to glow or blink in accordance with the amount of misalignment, thereby prompting the correction of the placement of the finger 12 on the placement position. At this point, the rows of visible light source elements 34-1 through 34-3 may be varied in lighting color. For example, the visible light source elements 34-1 may be lit in green, visible light source elements 34-2 in yellow, and visible light source elements 34-3 in red. Also, the rows of visible light source elements 34-1 through 34-3 may be lit and extinguished repeatedly one after another.

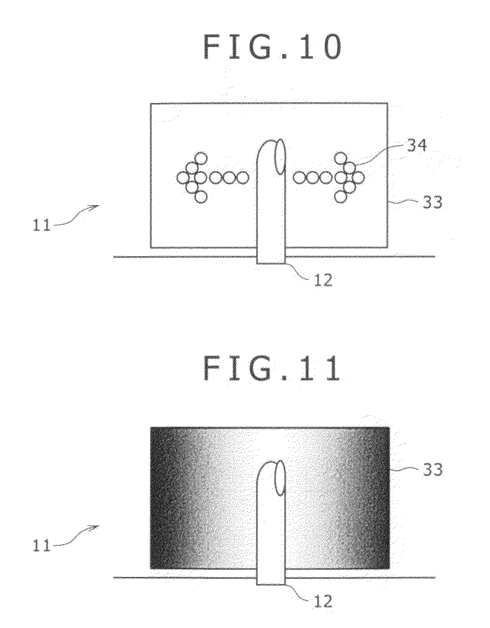

As shown in FIG. 10 for example, the light emission control portion 56 may cause the visible light source 34 to emit light in arrow shape in order to prompt the correction of the placement of the finger 12 on the placement position. The visible light source 34 shown in FIG. 10 may be caused to blink, vary in lighting color, or glow in a manner varying the length of the arrows in accordance with the misalignment amount fed from the misalignment amount computation portion 55.

The shapes and patterns of the light emission by the visible light source 34 as well as the lighting colors are not limited to those shown in FIGS. 9 and 10 as examples. Other suitable shapes and patterns of light emission as well as other lighting colors may be adopted instead.

Returning to the flowchart of FIG. 7, step S56 is followed by step S51 for another process.

Meanwhile, if it is determined in step S55 that the misalignment amount computed in step S54 is not larger than the predetermined threshold value, e.g., if it is determined that the actual misalignment amount and the relative misalignment amount are each not larger than the predetermined corresponding threshold value, then the misalignment amount computation portion 55 supplies the imaging control portion 52 with information representative of the misalignment amount (relative misalignment amount). Control is then transferred to step S57.

In step S57, the imaging control portion 52 adjusts the imaging parameters of the imaging section 32 in accordance with the information representative of the misalignment amount coming from the misalignment amount computation portion 55, and causes the imaging section 32 to take a finger image using the adjusted imaging parameters before feeding the acquired finger image to the registration/authentication processing portion 53. The adjustments allow the imaging section 32 to take the finger image that can be authenticated by the registration/authentication processing portion 53. If the actual misalignment amount and the relative misalignment amount are each zero (or approximately zero), then the imaging control portion 52 causes the acquired finger image to be sent to the registration/authentication processing portion 53 without adjusting the imaging parameters of the imaging section 32.

Subsequent to step S57, or if it is determined in step S52 that the imaged object is not a human finger, then control is transferred back to step S36 in the flowchart of FIG. 6.

Returning to the flowchart of FIG. 6, in step S37 following step S36, the registration/authentication processing portion 53 collates the finger image from the imaging section with the finger image of the user information retrieved from the registration database 36. At this point, depending on the result of the collation, the light emission control portion 56 may control the light emission of the visible light source 34 in a manner presenting the user with the outcome of the collation.

With the above steps carried out, if the user's finger is not aligned with the correct placement position during the authentication process based on the venous pattern of the finger and performed by the authentication unit with its finger placement position shaped flat, then an emission of the visible light source reflecting the amount of the finger's amount of misalignment with the placement position can be fed back to the user. The feedback allows the user to recognize that the finger is not aligned with the placement position. As a result, authentication can be performed accurately even where the placement position is shaped flat.

Whereas the foregoing description indicated that the amount of misalignment is calculated based on the received-light intensity and on the finger image, the misalignment amount may alternatively be computed from the received-light intensity alone.

In the foregoing paragraphs, the received-light intensity was shown to be computed based on the received-light level of the reflection of the visible light. Alternatively, the received-light intensity may be computed on the basis of the received-light level of the reflection of the near-infrared light emitted by the near-infrared light source 31.

Also in the foregoing paragraphs, the transmission filter 33 was shown to be structured simply to let near-infrared light and visible light pass through. Alternatively, a diffuser panel may be overlaid on the transmission filter 33.

FIG. 11 shows an example of the transmission filter 33 on which a diffuser panel is overlaid.

The diffuser panel overlaid on the transmission filter 33 diffuses the visible light emitted by the visible light source 34. This provides gradations at the placement position for the finger 12 as shown in FIG. 11. If the finger is not aligned with the placement position, the light emission of the visible light source 34 is controlled to vary the gradations in a manner prompting the correction of the placement of the finger 12 for alignment with the placement position.

Whereas FIG. 11 shows the example of the transmission filter 33 being overlaid with the diffuser panel, the transmission filter 33 may alternatively be structured to have a light-guiding material of a suitable shape embedded therein.

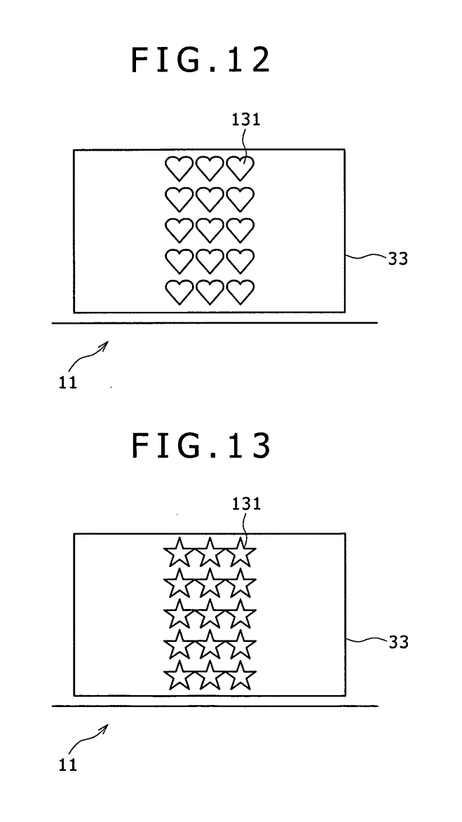

FIG. 12 shows an example of the transmission filter having light-guiding panels of an appropriate shape embedded therein. It is assumed that the transmission filter 33 shown in FIG. 12 is structured to inhibit the visible light of the visible light source 34 from passing through.

In FIG. 12, heart-shaped light-guiding materials 131 are embedded in the transmission filter 33 in a manner centering on the placement position. Because the transmission filter 33 blocks the visible light coming from the visible light source 34, the visible light of the visible light source 34 is introduced into the light-guiding materials 131 which in turn present the placement position of the finger 12 as shown in FIG. 12. If the finger 12 is not aligned with the placement position, the light emission of the visible light source 34 is controlled to vary the emission through the light-guiding materials 131 in a manner prompting the correction of the placement of the finger 12 for alignment with the placement position.

The shape of the light-guiding material 131 is not limited to the heart shape shown in FIG. 12. Alternatively, the light-guiding material 131 may be star-shaped as shown in FIG. 13. As other alternatives, the light-guiding material 131 may obviously take various other shapes including an equilateral triangle or a square.

As described above, when the diffuser panel is overlaid on the transmission filter 33 or the light-guiding materials are embedded therein, it is possible visibly to vary the manner in which the finger placement position is presented or the kind of feedback with which the user is notified of finger misalignment from the placement position.

The authentication unit 11 in FIG. 2 was shown to have the near-infrared light source 31, imaging section 32, visible light source 34, and light-receiving sensor 35 mounted on the same substrate under the transmission filter 33. Alternatively, the registration database 36 and control section 37 may also be mounted on the same substrate to make the authentication unit 11 thinner than ever in shape.

In the foregoing paragraphs, the emission of the visible light source 34 was shown to be given as the feedback to the user where the finger is not aligned with the placement position. Alternatively, the authentication unit 11 may be structured to incorporate or connect with a display section for displaying predetermined images or text and a sound output section for outputting predetermined sounds. The display section and the sound output section may then be arranged to give a suitable display and output suitable sounds as the feedback to the user.

### [Another Typical Functional Structure of the Authentication Unit]

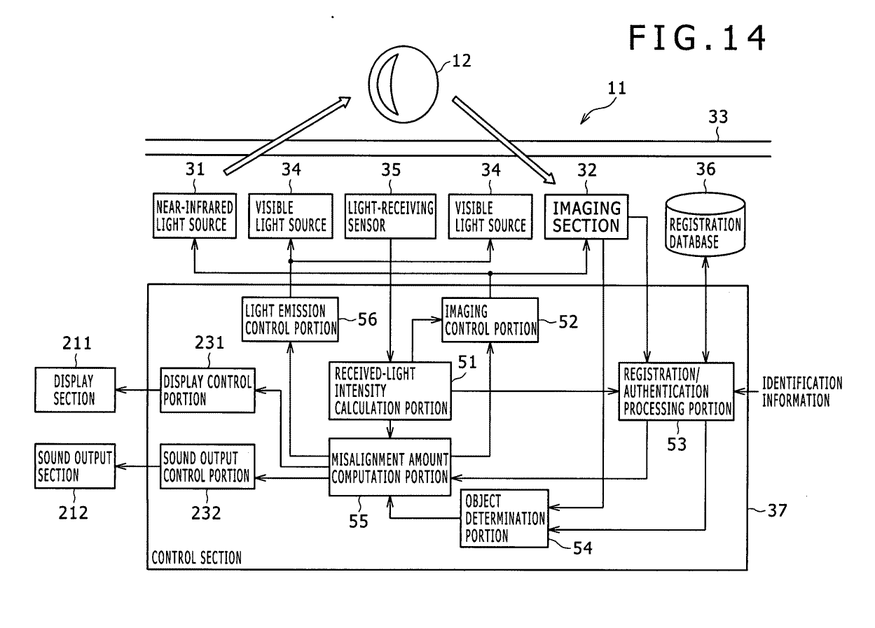

Explained below in reference to FIG. 14 is another typical functional structure of the authentication unit 11 that causes the display section to give a display and the sound output section to output sounds as the feedback to the user.

The authentication unit 11 in FIG. 14 is made up of a near-infrared light source 31, an imaging section 32, a transmission filter 33, a visible light source 34, a light-receiving sensor 35, a registration database 36, a control section 37, a display section 211, and a sound output section 212.

In the authentication unit 11 of FIG. 14, the components functionally equivalent to those already shown in the authentication unit 11 of FIG. 2 are designated by like reference names and like reference numerals, and their descriptions are omitted hereunder.

That is, the difference between the authentication unit 11 in FIG. 14 and its counterpart in FIG. 2 is that the display section 211 and sound output section 212 are additionally provided.

The display section 211 is composed of a display device such as a liquid crystal display (LCD) or an organic electroluminescence (EL) display. Under control of the control section 37, the display section 211 displays predetermined images or text.

The sound output section 212 is composed of so-called speakers that output predetermined sounds under control of the control section 37.

The control section 37 in FIG. 14 is made up of a received-light intensity calculation portion 51, an imaging control portion 52, a registration/authentication processing portion 53, an object determination portion 54, a misalignment amount computation portion 55, a light emission control portion 56, a display control portion 231, and a sound output control portion 232.

In the control section 37 of FIG. 14, the components functionally equivalent to those already shown in the control section 37 of FIG. 2 are designated by like names and like reference numerals, and their descriptions are omitted hereunder.

That is, the difference between the control section in FIG. 14 and its counterpart in FIG. 2 is that the display control portion 231 and sound output control portion 232 are additionally provided.

The display control portion 231 controls the display of the display section 211 in a manner prompting the correction of the placement of the finger 12 for alignment with the placement position in accordance with the misalignment amount supplied from the misalignment amount computation portion 55.

The sound output control portion 232 controls the sound output of the sound output section 212 in a manner prompting the correction of the placement of the finger 12 for alignment with the placement position in accordance with the misalignment amount supplied from the misalignment amount computation portion 55.

The user registration process and the user authentication process performed by the authentication unit 11 in FIG. 14 are the same as the user registration process and the user authentication process which are carried out by the authentication unit 11 in FIG. 2 and which were described above in reference to the flowcharts of FIGS. 4 and 5. The descriptions of these processes are thus omitted hereunder.

Also, the misalignment notification process performed by the authentication unit 11 in FIG. 14 is basically the same as the misalignment notification process which is carried out by the authentication unit 11 in FIG. 2 and which was described above in reference to the flowchart of FIG. 7. The description of this process is therefore omitted hereunder.

It should be noted, however, that in the misalignment notification process performed by the authentication unit 11 of FIG. 14, the misalignment amount computation portion 55 supplies the display control portion 231 with information representative of the computed misalignment amount (actual misalignment amount) if the misalignment amount is determined to be larger than the predetermined threshold value in step S55 of FIG. 7. And in step S56, in response to the information representing the misalignment amount coming from the misalignment amount computation portion 55, the display control portion 231 controls the display of the display section 211 in a manner prompting the correction of the placement of the finger 12 for alignment with the placement position.

More specifically, depending on the actual misalignment amount, the display section 211 is caused typically to display an arrow image or a text such as “Move your finger by _ mm to the right (left)” thereby prompting the correction of the placement of the finger 12 for alignment with the placement position.

After the above-described misalignment notification process, the display control portion 231 may control the display of the display section 211 in accordance with the result of the collation in a manner presenting the user with the outcome of the collation.

Also in the misalignment notification process performed by the authentication unit 11 of FIG. 14, the misalignment amount computation portion 55 may alternatively supply the sound output control portion 232 with the information representative of the computed misalignment amount (actual misalignment amount) if the misalignment amount is determined to be larger than the predetermined threshold value in step S55 of FIG. 7. Then in step S56, in accordance with the information representing the misalignment amount coming from the misalignment amount computation portion 55, the sound output control portion 232 controls the sound output of the sound output section 212 in a manner prompting the correction of the placement of the finger 12 for alignment with the placement position.

More specifically, depending on the actual misalignment amount, the sound output section 212 is caused typically to sounds such as “Move your finger by mm to the right (left)” in order to prompt the correction of the placement of the finger 12 for alignment with the placement position.

After the above-described misalignment notification process, the sound output control portion 232 may control the sound output of the sound output section 212 in accordance with the result of the collation in a manner presenting the user with the outcome of the collation.

In the above-described authentication process based on the venous pattern of the finger and performed by the authentication unit with its finger placement position shaped flat, the display or the sound reflecting any misalignment of the user's finger with the placement position is presented to the user as the feedback indicating the misalignment. This allows the user to recognize that his or her finger is not aligned with the placement position. As a result, authentication can be performed accurately even where the placement position is shaped flat.

Also, the authentication unit 11 in FIG. 14 was shown to have the near-infrared light source 31, imaging section 32, visible light source 34, and light-receiving sensor 35 mounted on the same substrate under the transmission filter 33. Alternatively, the registration database 36 and control section 37 may also be mounted on the same substrate to make the authentication unit 11 thinner than ever in shape.

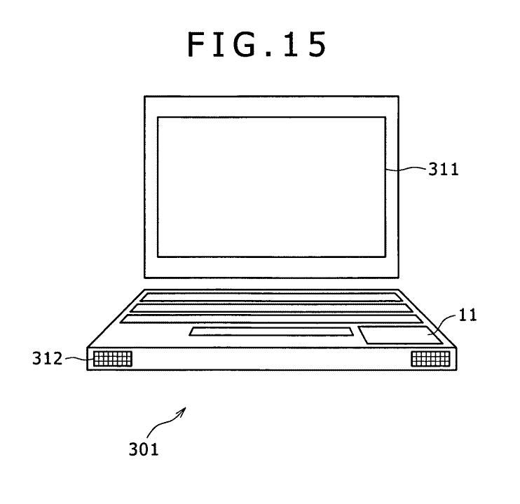

Furthermore, because the authentication unit 11 of FIG. 14 has its body shaped flat on the side of the finger 12 (i.e., topside of the transmission filter 33), the unit 11 can be incorporated in a notebook-size personal computer 301 shown in FIG. 15 or in other folding portable terminal equipment.

In the personal computer 301 shown in FIG. 15, a display section 311 and a sound output section 312 correspond respectively to the display section 211 and sound output section 211 that were explained above in reference to FIG. 14.

Also in FIG. 15, the authentication unit 11 is shown mounted on the surface of the body of the personal computer 301 (i.e., enclosure corresponding to the display section 311). Alternatively, by taking advantage of its thin shape, the authentication unit 11 may be furnished as a retractable sliding part that can slide into and out of a side of the body of the personal computer 301.

The above structure allows the personal computer 301 as a whole including its authentication facility to be shaped thinner than ever before.

In the foregoing paragraphs, the display of the display section or the sound output of the sound output section was shown to be presented to the user as the feedback indicating any misalignment of the finger with the placement position. Alternatively, a temperature difference or vibrations near the placement position may be given to the user as the feedback indicative of the misalignment.

### [Another Typical Functional Structure of the Authentication Unit]

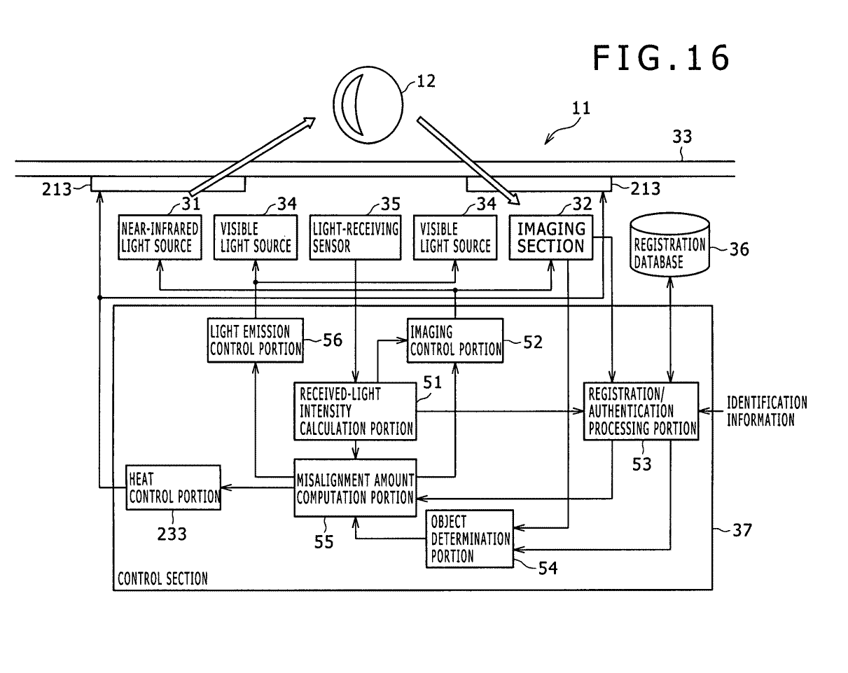

Explained below in reference to FIG. 16 is a further typical functional structure of the authentication unit 11 that provides the user with a temperature difference near the finger placement position as the feedback to the user.

The authentication unit 11 in FIG. 16 is made up of a near-infrared light source 31, an imaging section 32, a transmission filter 33, a visible light source 34, a light-receiving sensor 35, a registration database 36, a control section 37, and a heating element 213.

In the authentication unit 11 of FIG. 16, the components functionally equivalent to those already shown in the authentication unit 11 of FIG. 2 are designated by like names and like reference numerals, and their descriptions are omitted hereunder.

That is, the difference between the authentication unit 11 in FIG. 16 and its counterpart in FIG. 2 is that the heating element 213 is additionally provided.

The heating element 213 is structured to be a thin metal sheet enveloped in plastic resin film. As such, the heating element 213 is attached to positions away from the finger placement position by a predetermined distance (e.g., positions corresponding to the visible light source element 34-3 in FIG. 9) under the bottom side of the transmission filter 33. The heating element 213 generates heat when an electrical current is allowed to flow through the metal sheet under control of the control section 37, whereby a temperature difference is produced on the transmission filter 33.

The control section 37 in FIG. 16 is made up of a received-light intensity calculation portion 51, an imaging control portion 52, a registration/authentication processing portion 53, an object determination portion 54, a misalignment amount computation portion 55, a light emission control portion 56, and a heat control portion 233.

In the control section 37 of FIG. 16, the components functionally equivalent to those already shown in the control section 37 of FIG. 2 are designated by like names and like reference numerals, and their descriptions are omitted hereunder.

That is, the difference between the control section 37 in FIG. 16 and its counterpart in FIG. 2 is that the heat control portion 233 is additionally provided.

In accordance with the misalignment amount coming from the misalignment amount computation portion 55, the heat control portion 233 controls the heating of the heating element 213 in a manner prompting the correction of the placement of the finger 12 for alignment with the placement position.

The user registration process and the user authentication process performed by the authentication unit 11 in FIG. 16 are the same as the user registration process and the user authentication process which are carried out by the authentication unit 11 in FIG. 2 and which were described above in reference to the flowcharts of FIGS. 4 and 5. The descriptions of these processes are thus omitted hereunder.

Also, the misalignment notification process performed by the authentication unit 11 in FIG. 16 is basically the same as the misalignment notification process which is carried out by the authentication unit 11 in FIG. 2 and which was described above in reference to the flowchart of FIG. 7. The description of this process is therefore omitted hereunder.

It should be noted, however, that in the misalignment notification process performed by the authentication unit 11 of FIG. 16, the misalignment amount computation portion 55 supplies the heat control portion 233 with information representative of the computed misalignment amount (actual misalignment amount) if the misalignment amount is determined to be larger than the predetermined threshold value in step S55 of FIG. 7. And in step S56, in response to the information representing the misalignment amount coming from the misalignment amount computation portion 55, the heat control portion 233 controls the heating of the heating element 213 in a manner prompting the correction of the placement of the finger 12 for alignment with the placement position.

More specifically, an electrical current reflecting the actual misalignment amount is allowed to flow through the metal sheet of the heating element 213. This causes the misaligned position away from the placement position on the transmission filter 33 to generate heat reflecting the misalignment amount in a manner prompting the correction of the placement of the finger 12 for alignment with the placement position. At this point, the temperature at the placement position on the transmission filter 33 is different from (i.e., lower than) the temperature at the misaligned position away from the placement position, so that the user can recognize the low-temperature position to be the correct placement position.

In the above-described structure, the misaligned position away from the finger placement position was shown to have a raised temperature (i.e., heated in proportion to the amount of misalignment with the placement position). The point, however, is that the temperature difference between the placement position and the misaligned position need only be recognized by the user. In that sense, the misaligned position away from the placement position may be arranged alternatively to have a lower temperature than the placement position (i.e., heat is absorbed in accordance with the amount of misalignment with the placement position).

### [Another Typical Functional Structure of the Authentication Unit]

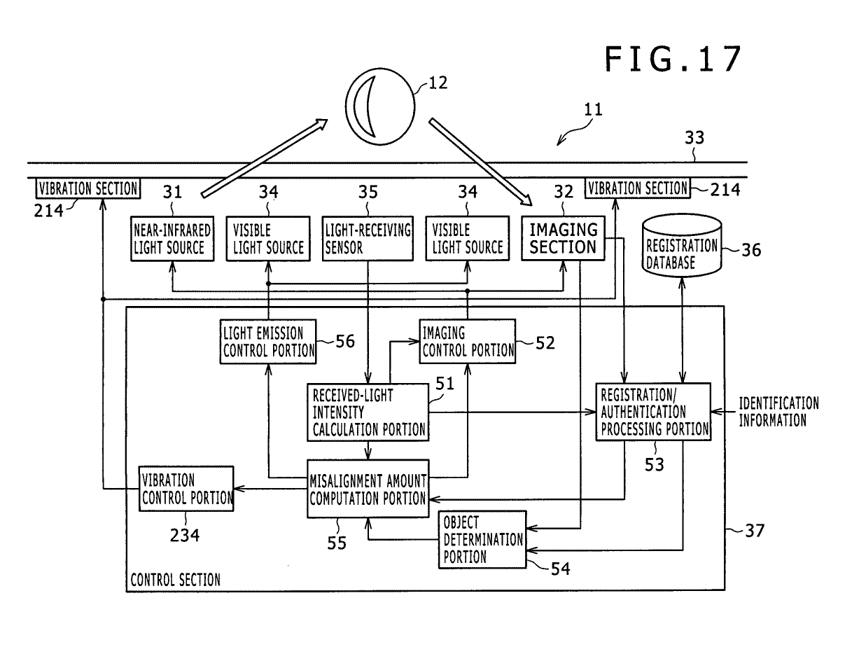

Explained below in reference to FIG. 17 is an even further typical functional structure of the authentication unit 11 that provides the user with vibrations near the placement position as the feedback.

The authentication unit 11 in FIG. 17 is made up of a near-infrared light source 31, an imaging section 32, a transmission filter 33, a visible light source 34, a light-receiving sensor 35, a registration database 36, a control section 37, and a vibration section 214.

In the authentication unit 11 of FIG. 17, the components functionally equivalent to those already shown in the authentication unit 11 of FIG. 2 are designated by like names and like reference numerals, and their descriptions are omitted hereunder.

That is, the difference between the authentication unit 11 in FIG. 17 and its counterpart in FIG. 2 is that the vibration section 214 is additionally provided.

The vibration section 214 may be structured to include a small-sized motor equipped with an eccentric weight. The vibration section 214 is attached onto that position of the bottom side of the transmission filter 33 which is displaced by a predetermined distance from the finger placement position (e.g., onto the position corresponding to the visible light source element 34-3 in FIG. 9). Under control of the control section 37, the vibration section 214 generates vibrations causing part or the entire transmission filter 33 to vibrate.

The control section 37 in FIG. 17 is made up of a received-light intensity calculation portion 51, an imaging control portion 52, a registration/authentication processing portion 53, an object determination portion 54, a misalignment amount computation portion 55, a light emission control portion 56, and a vibration control portion 234.

In the control section 37 of FIG. 17, the components functionally equivalent to those already shown in the control section 37 of FIG. 2 are designated by like names and like reference numerals, and their descriptions are omitted hereunder.

That is, the difference between the control section in FIG. 17 and its counterpart in FIG. 2 is that the vibration control portion 234 is additionally provided.

In accordance with the misalignment amount coming from the misalignment amount computation portion 55, the vibration control portion 234 controls the vibration of the vibration section 214 in a manner prompting the correction of the placement of the finger 12 for alignment with the placement position.

The user registration process and the user authentication process performed by the authentication unit 11 in FIG. 17 are the same as the user registration process and the user authentication process which are carried out by the authentication unit 11 in FIG. 2 and which were described above in reference to the flowcharts of FIGS. 4 and 5. The descriptions of these processes are thus omitted hereunder.

Also, the misalignment notification process performed by the authentication unit 11 in FIG. 17 is basically the same as the misalignment notification process which is carried out by the authentication unit 11 in FIG. 2 and which was described above in reference to the flowchart of FIG. 7. The description of this process is therefore omitted hereunder.

It should be noted, however, that in the misalignment notification process performed by the authentication unit 11 of FIG. 17, the misalignment amount computation portion 55 supplies the vibration control portion 234 with information representative of the computed misalignment amount (actual misalignment amount) if the misalignment amount is determined to be larger than the predetermined threshold value in step S55 of FIG. 7. And in step S56, in response to the information representing the misalignment amount coming from the misalignment amount computation portion 55, the vibration control portion 234 controls the vibration of the vibration section 214 in a manner prompting the correction of the placement of the finger 12 for alignment with the placement position.

More specifically, the vibration section 214 causes the transmission filter 33 to vibrate the misaligned position away from the finger placement position at a magnitude (or with a pattern) reflecting the actual misalignment amount so as to prompt the correction of the placement of the finger 12 for alignment with the placement position. The vibrations near the placement position on the transmission filter 33 are felt smaller than those at a significantly misaligned position away from the placement position. This allows the user to recognize that the smaller the magnitude of the vibrations, the closer the finger position to the placement position.

In the above-described structure, the misaligned position was shown to vibrate more the farther away from the finger placement position (i.e., the placement position does not vibrate). The point, however, is that the difference in (or the absence of) the magnitude of vibrations need only be felt by the user between the placement position and the misaligned position. In that sense, there may be provided an alternative structure whereby the farther away from the placement position, the smaller the vibrations felt at the misaligned position of the finger (i.e., the placement position vibrates the most).

In the above-described authentication process based on the venous pattern of the finger and performed by the authentication unit with its finger placement position shaped flat, the temperature difference or the vibration reflecting any misalignment of the user's finger with the placement position is presented to the user as the feedback indicating the misalignment. This allows the user to recognize that his or her finger is not aligned with the placement position. As a result, authentication can be performed accurately even where the placement position is shaped flat.

In the foregoing paragraphs, the temperature difference or the vibration was shown given to the user as the feedback indicating any misalignment of his or her finger with the placement position. Alternatively, the above-described emission of visible light, display, sound, temperature difference, and vibration may all be given to the user as the feedback indicative of any misalignment.

### [Another Typical Functional Structure of the Authentication Unit]

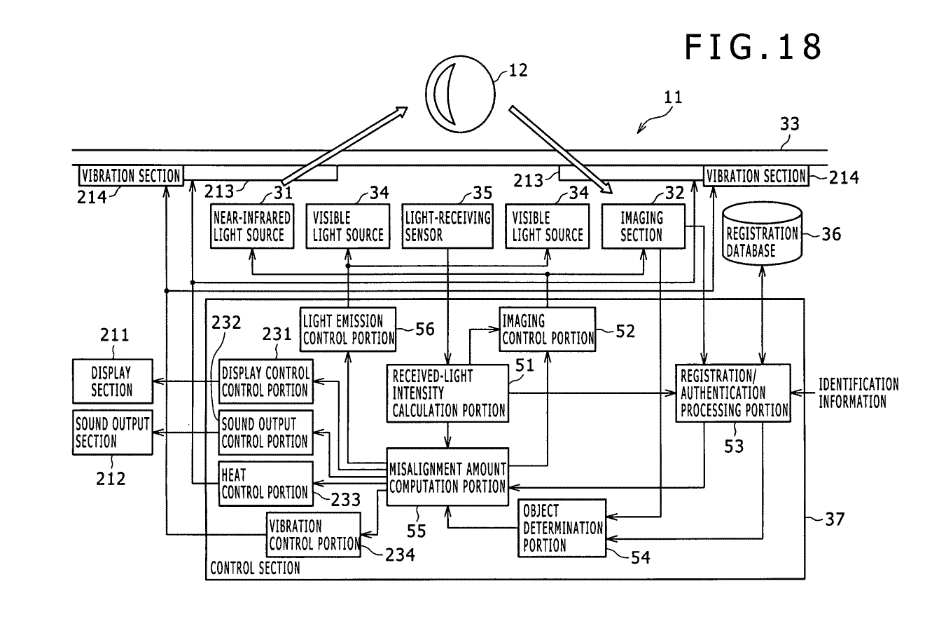

Explained below in reference to FIG. 18 is a still further typical functional structure of the authentication unit 11 that provides the user with all of the emission of visible light, display, sound, temperature difference, and vibration as the feedback to the user.

In the authentication unit 11 of FIG. 18, the components functionally equivalent to those already found in the authentication unit 11 indicated in FIG. 2, 14, 16 or 17 are designated by like names and like reference numerals, and their descriptions are omitted hereunder.

Also, the user registration process, user authentication process, and misalignment notification process performed by the authentication unit 11 in FIG. 18 are the same as those described above, and thus will not be discussed further.

In the above-described authentication process based on the venous pattern of the finger and performed by the authentication unit with its finger placement position shaped flat, the emission of visible light, display, sound, temperature difference, and vibration reflecting any misalignment of the user's finger with the placement position may all be presented to the user as the feedback indicating the misalignment. This allows the user to recognize that his or her finger is not aligned with the placement position. As a result, authentication can be performed accurately even where the placement position is shaped flat.

In the foregoing paragraphs, all of the emission of visible light, display, sound, temperature difference, and vibration were shown given to the user as the feedback indicating any misalignment of his or her finger with the placement position. Alternatively, at least two items out of the above-described emission of visible light, display, sound, temperature difference, and vibration may be given in combination to the user as the feedback indicative of any misalignment. This also allows the user to recognize any misalignment of his or her finger with the placement position more unambiguously than ever.

In the foregoing description, the present invention was explained as applicable to the authentication unit performing the authentication process by utilizing the veins of the human finger. Alternatively, the invention may be applied to diverse authentication units including those that carry out authentication processes by use of part of the veins of the human body, such as the veins of the palm.

It should be understood by those skilled in the art that various modifications, combinations, sub-combinations and alterations may occur depending on design requirements and other factor in so far as they are within the scope of the appended claims or the equivalents thereof.

What is claimed is:

1. An information processing apparatus for performing authentication using veins of a living body part, said information processing apparatus comprising:
a visible light source for presenting through light emission the position on which to place said living body part;
light-receiving means for receiving reflected light of said visible light from said visible light source;
computation means for computing the amount of misalignment of said living body part with the placement position on the basis of the intensity of said reflected light received by said light-receiving means; and
control means for prompting correction of the placement of said living body part for alignment with said placement position in accordance with the misalignment amount computed by said computation means.

2. The information processing apparatus according to claim 1, wherein said control means prompts the correction of the placement of said living body part for alignment with said placement position by controlling the emission of said visible light in accordance with said misalignment amount computed by said computation means.

3. The information processing apparatus according to claim 2, further comprising:
a near-infrared light source for emitting near-infrared light to said living body part; and
imaging means for taking an image of said living body part to which said near-infrared light is emitted;
wherein said computation means computes said misalignment amount based on the intensity of said reflected light received by said light-receiving means and on the image of said living body part taken by said imaging means.

4. The information processing apparatus according to claim 3, wherein, if said misalignment amount is larger than a predetermined threshold value, then said control means prompts the correction of the placement of said living body part for alignment with said placement position.

5. The information processing apparatus according to claim 4, further comprising imaging control means for adjusting imaging parameters of said imaging means if said misalignment amount is smaller than said predetermined threshold value.

6. The information processing apparatus according to claim 3, further comprising determination means for determining whether an object imaged by said imaging means is said living body part;
wherein, if said determination means determines that said object is said living body part, then said computation means computes said misalignment amount.

7. The information processing apparatus according to claim 3, further comprising recording means for recording said image taken by said imaging means upon user registration;
wherein said computation means computes said misalignment amount based on the intensity of said reflected light received by said light-receiving means and on a difference between the image taken by said imaging means and the image recorded in said recording means.

8. The information processing apparatus according to claim 1, further comprising display means for displaying a predetermined image or text;
wherein said control means causes said display means to display an image or a text prompting the correction of the placement of said living body part for alignment with said placement position in accordance with said misalignment amount computed by said computation means.

9. The information processing apparatus according to claim 1, further comprising sound output means for outputting a sound;
wherein said control means causes said sound output means to output a sound prompting the correction of the placement of said living body part for alignment with said placement position in accordance with said misalignment amount computed by said computation means.

10. The information processing apparatus according to claim 1, further comprising temperature difference generation means for generating a temperature difference near said placement position;
wherein said control means causes said temperature difference generation means to generate a temperature difference prompting the correction of the placement of said living body part for alignment with said placement position in accordance with said misalignment amount computed by said computation means.

11. The information processing apparatus according to claim 1, further comprising vibration generation means for generating vibrations near said placement position;
wherein said control means causes said vibration generation means to generate vibrations prompting the correction of the placement of said living body part for alignment with said placement position in accordance with said misalignment amount computed by said computation means.

12. The information processing apparatus according to claim 1, further comprising:
display means for displaying a predetermined image or text;
sound output means for outputting a sound;
temperature difference generation means for generating a temperature difference near said placement position; and
vibration generation means for generating vibrations near said placement position;
wherein said control means causes said visible light source to emit the light prompting the correction of the placement of said living body part for alignment with said placement position in accordance with said misalignment amount computed by said computation means,
said control means causes said display means to display an image or a text prompting the correction of the placement of said living body part for alignment with said placement position in accordance with said misalignment amount,
said control means causes said sound output means to output a sound prompting the correction of the placement of said living body part for alignment with said placement position in accordance with said misalignment amount,
said control means causes said temperature difference generation means to generate a temperature difference prompting the correction of the placement of said living body part for alignment with said placement position in accordance with said misalignment amount, and
said control means causes said vibration generation means to generate vibrations prompting the correction of the placement of said living body part for alignment with said placement position in accordance with said misalignment amount.

13. The information processing apparatus according to claim 1, wherein said living body part is a human finger.

14. An information processing method for use with an information processing apparatus which performs authentication using veins of a living body part and which includes a visible light source for presenting through light emission the position on which to place said living body part and light-receiving means for receiving reflected light of said visible light from said visible light source, said information processing method comprising the steps of:
computing the amount of misalignment of said living body part with the placement position on the basis of the intensity of said reflected light received by said light-receiving means; and
performing control to prompt correction of the placement of said living body part for alignment with said placement position in accordance with the misalignment amount computed by said computation means.
